(19) United States
(12) Patent Application Publication
(10) Pub. No.: US 2022/0236206 A1
(43) Pub. Date: Jul. 28, 2022

(54) BIOSENSOR FOR DETERMINATION OF HEMOGLOBIN

(71) Applicant: Trividia Health, Inc., Fort Lauderdale, FL (US)

(72) Inventors: John Pasqua, Delray Beach, FL (US); Janette Lackore, Weston, FL (US); Jessica Gendron, Margate, FL (US); Hernan R. Rengifo, Miramar, FL (US); Ngoc Minh Phuong Bui, Coconut Creek, FL (US); Gaganbir Johal, Boca Raton, FL (US)

(21) Appl. No.: 17/584,182

(22) Filed: Jan. 25, 2022

Related U.S. Application Data
(60) Provisional application No. 63/141,310, filed on Jan. 25, 2021.

(51) Int. Cl. G01N 27/07; B01L 3/00; G01N 33/49

(52) U.S. Cl. CPC G01N 27/07; B01L 3/502715; B01L 2300/0825; B01L 2300/0645; G01N 33/492

(57) ABSTRACT

The present disclosure provides a test strip including: a conductive pattern formed on a substrate, the conductive pattern being formed from a thin film material, the conductive pattern including: a plurality of electrodes configured to perform a reagent-free measurement of hematocrit levels in a blood sample; a plurality of conductive contacts configured to communicate with a test meter; and a plurality of conductive traces configured to electrically connect the plurality of electrodes to the plurality of conductive contacts; an inert layer positioned on at least a portion of the conductive pattern; and a capillary chamber exposing at least a portion of the plurality of electrodes, the capillary chamber being defined by the inert layer for receiving a blood sample and delivering the blood sample to the plurality of electrodes.

### RELATED APPLICATIONS

The present application claims priority to and the benefit of U.S. Provisional Application No. 63/141,310, filed Jan. 25, 2021, which is incorporated herein by reference in its entirety.

### FIELD

The present disclosure relates to a reagent-free test strip or test strip with an inert-coating suitable for determination of a target substance. In particular, the present disclosure relates to a reagent-free test strip or test strip with an inert-coating including use of thin layer noble metal and/or non-noble metal alloy electrodes for the determination of hematocrit/hemoglobin.

### BACKGROUND

Generally, colorimetric methods for determining hemoglobin in capillary, venous, and/or arterial blood are very common and often rely on optical measurement of chemically stable compound(s) formed by a reagent-based reaction. Common examples of colorimetric methods include Vanzetti's Azide Methemoglobin method, Sahli's Method, and Hemoglobincyanide Method. Reagent-free colorimetric measurements are also common and utilize microcuvettes, which require precise optical quality cuvette molding for the consumable. In addition, reagent based microcuvettes used in photometric and/or electrochemical measurement of hemoglobin or hematocrit often require use of lysing reagents and/or oxidants which may impact product stability. Additionally, hemoglobin and hematocrit measurement methods often have manufacturability and shelf life constraints. Common techniques of measuring hematocrit, such as conductivity, often lead to inaccurate measurements because of sensitivity to varying electrolytes and protein concentrations in blood. Therefore, it would be advantageous to develop a stable test strip that is suitable for mass production with accurate performance.

### SUMMARY

There is a need for improvements for measuring hematocrit/hemoglobin using a reagent free and/or inert coated test strip. The present disclosure is directed toward further solutions to address this need, in addition to having other desirable characteristics.

In some aspects, the present disclosure provides a test strip including: a conductive pattern formed on a substrate, the conductive pattern being formed from a thin film material, the conductive pattern including: a plurality of electrodes configured to perform a reagent-free measurement of hematocrit levels in a blood sample; a plurality of conductive contacts configured to communicate with a test meter; and a plurality of conductive traces configured to electrically connect the plurality of electrodes to the plurality of conductive contacts; an inert layer positioned on at least a portion of the conductive pattern; and a capillary chamber exposing at least a portion of the plurality of electrodes, the capillary chamber being defined by the inert layer for receiving a blood sample and delivering the blood sample to the plurality of electrodes.

In some aspects, the present disclosure relates to a system for measuring hematocrit in a blood sample, the system including: a test strip including: a conductive pattern formed on a substrate, the conductive pattern being formed from a thin film material, the conductive pattern including a plurality of electrodes configured to perform a reagent-free measurement of hematocrit levels in a blood sample; a plurality of conductive contacts; and a plurality of conductive traces configured to electrically connect the plurality of electrodes to the plurality of conductive contacts; an inert layer positioned on at least a portion of the conductive pattern; a capillary chamber exposing at least a portion of the plurality of electrodes, the capillary chamber being defined by the inert layer for receiving a blood sample and delivering the blood sample to the plurality of electrodes; and a test meter configured to accept the test strip and to connect to the plurality of conductive contacts to determine a level of hematocrit in the blood sample received on the test strip.

In some aspects, the test meter is configured to apply an AC impedance at a plurality of frequencies across the plurality of electrodes. In some aspects, test meter is configured to apply a low voltage less than 100 mv signal across the plurality of electrodes. In some aspects, the test meter is further configured to determine a hemoglobin value from the level of hematocrit in the blood sample.

In some aspects, the plurality of electrodes are uniform thin film electrodes. In some aspects, the plurality of electrodes has a thickness in a range of 10 nm (100 Å) to 3,000 nm (3 μm). In some aspects, the plurality of electrodes has a thickness in a range of 20 nm (200 Å) to 1,000 nm (1 μm). In some aspects, the plurality of electrodes has a thickness in a range of 30 nm (300 Å) to 60 nm (600 Å). In some aspects, the plurality of electrodes are formed from a thin film of a non-noble metal. In some aspects, the plurality of electrodes includes a proximal electrode and a distal electrode, wherein a distance between proximal electrode and the distal electrode is in a range from 0.5 mm-5.5 mm. In some aspects, the inert layer fully coats the plurality of electrodes.

In some aspects, the present disclosure provides a method for determining a hematocrit value in a blood sample, the method including: applying, by a test meter, an electrical current across a plurality of electrodes on a test strip, wherein the test strip includes a conductive pattern formed on a substrate, the conductive pattern being formed from a thin film material, the conductive pattern including the plurality of electrodes configured to perform a reagent-free measurement of hematocrit levels in a blood sample; a plurality of conductive contacts; and a plurality of conductive traces configured to electrically connect the plurality of electrodes to the plurality of conductive contacts; an inert layer positioned on at least a portion of the conductive pattern; a capillary chamber exposing at least a portion of the plurality of electrodes, the capillary chamber being defined by the inert layer for receiving a blood sample and delivering the blood sample to the plurality of electrodes; measuring, by the test meter, conductivity of the blood sample; and calculating, by the test meter, a hematocrit value of the blood sample based on the conductivity of the blood sample. In some aspects, the method further includes a step of determining, by the test meter, a hemoglobin value of the blood sample from the hematocrit value.

### BRIEF DESCRIPTION OF THE DRAWINGS

These and other characteristics of the present disclosure will be more fully understood by reference to the following detailed description in conjunction with the attached drawings, in which:

FIG. 1A is an illustrative isometric view of a test strip, in accordance with the present disclosure;

FIG. 1B is an illustrative exploded view of a test strip, in accordance with the present disclosure;

FIG. 1C illustrates a cross-sectional view of a test strip, in accordance with the present disclosure;

FIGS. 2A and 2B illustrate a meter according to some embodiments of the present disclosure;

FIG. 3A is a chart showing a hematocrit bias from reference over time, in accordance with the present disclosure;

FIG. 3B is a chart showing a linearity response between hemoglobin results over time, in accordance with the present disclosure;

FIG. 4A is a chart showing hematocrit measurement using a test strip, in accordance with the present disclosure; and

FIG. 4B is a chart showing hemoglobin determination using a test strip, in accordance with the present disclosure.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

An illustrative embodiment of the present disclosure relates to systems and methods to produce a reagent-free test strip or test strip with an inert-coating constructed from a combination of metals, non-noble metals, and/or alloys. The reagent-free test strip or test strip with an inert-coating can be used to accurately measure hematocrit and/or hemoglobin levels within a sample, such as in blood or plasma utilizing a variety of techniques. The test strip or biosensor of the present disclosure can be used for testing at home, at blood and/or plasma donation centers, hospitals, clinics, point-of-care, ambulatory/first responders, veterinary, and/or similar markets.

In accordance with example embodiments of the present disclosure, a device for the measurement of hematocrit in a human blood sample is provided. The device includes using reagent free test strips or test strips with an inert coating capable of obtaining an electrical measurement using low voltage. In some embodiments, the voltage needed for the electrical measurement is less than 100 mV.

In accordance with aspects of the present disclosure, the device can include test strips with a plurality of electrical uniform, thin film electrodes. The device can make use of known correlation of hematocrit to hemoglobin relationships to determine the hemoglobin concentration in the blood sample. The electrical measurement can be an AC impedance measurement. The electrical measurement can be an AC impedance at a plurality of frequencies.

In accordance with aspects of the present disclosure, the electrodes can be composed of any of noble metal, non-noble metal alloys, and non-metal. The electrode film thickness can be nanometer to micrometer in size. For example, the thickness range for the electrodes can be 10 nm (100 Å) to 3,000 nm (3 μm). In some embodiments, the thickness is 20 nm (200 Å) to 1,000 nm (1 μm). In some embodiments, the thickness is 30 nm (300 Å) to 60 nm (600 Å). The electrodes can have a distance D between proximal and distal electrode(s) of about 0.5 mm to about 5.5 mm. The electrodes can include an inert coating that only partially coats or fully coats the test strip chamber/electrodes. The inert coating can include of a surfactant and/or polymer.

In accordance with example embodiments of the present disclosure, a device for the measurement of hematocrit in a non-human blood sample is provided. The device includes using reagent free test strips or test strips with an inert coating.

In the present disclosure, the determination of hematocrit on the reagent-free, thin-film test strip can be used with a variety of common techniques/meters that can be driven by very low voltages to provide very accurate and precise measurements. For example, techniques such as AC impedance, a DC charging current, conductivity, etc. can be used with the test strip of the present disclosure to measure hematocrit/hemoglobin. Selection of the techniques, the type of thin-film electrode substrate, strip storage conditions, and use of inert coatings and/or materials can be adjusted to influence accuracy, precision, and/or stability of a test strip over a wide range of hematocrit or hemoglobin levels. Certain combinations of electrode substrate type and/or strip storage conditions, coupled with the strip performance characteristics, such as strip stability, can significantly improve with inert coating and/or electrode surface modification.

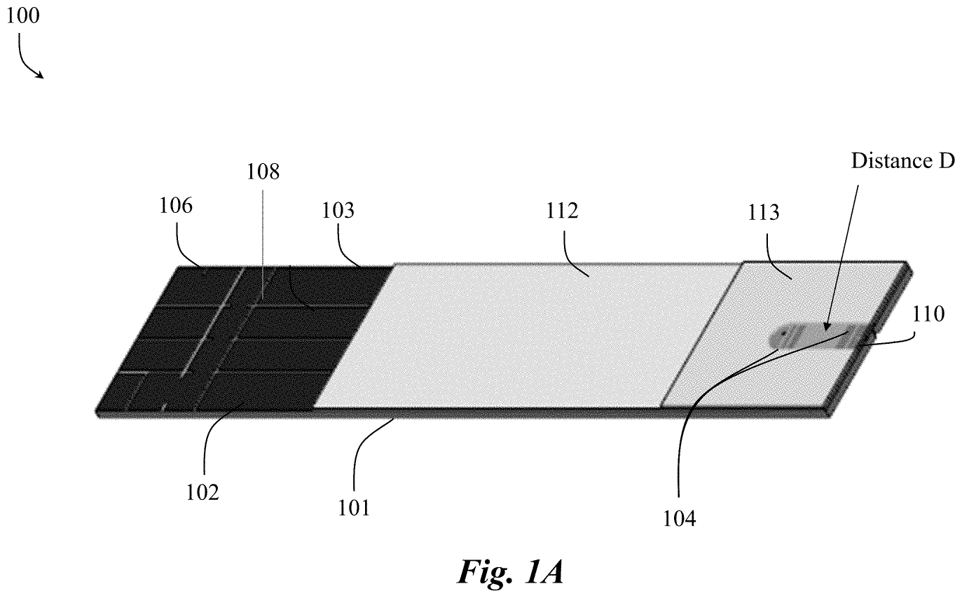

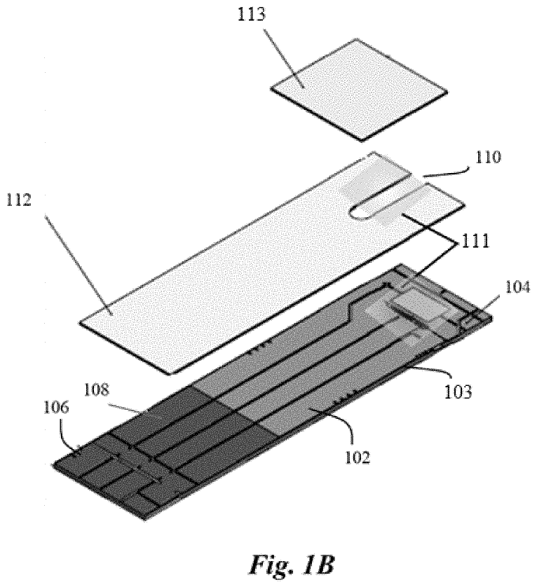

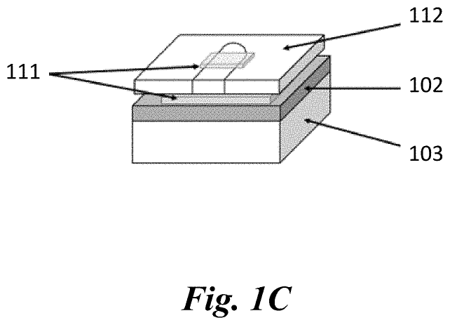

FIGS. 1A through 4B illustrate an example embodiment or embodiments of improved operation for a test strip or biosensor for measuring hematocrit and/or hemoglobin, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

Referring to FIG. 1A, FIG. 1B and FIG. 1C, in some embodiments, a reagent-free biosensor 100 (or test strip) can be designed without having a reagent or any other chemicals on the test strip to measure hematocrit using a test meter. It will be understood that the test strip does not include any reagent in any form in order to perform any measurements, including but not limited to hematocrit measurements and hemoglobin measurements. All measurements described herein can be performed without the use of a reagent. In some embodiments, the test strips of the present disclosure only include electrodes without a reagent. In other words, none of the electrodes on the test strip of the present disclosure include a reagent. In some embodiments, such reagent free design enables a simpler and less costly design for a test strip. In some embodiments, because the test strips of the present disclosure are reagent free, they are easier to manufacture. In some embodiments, the biosensor 100 can have a base layer 101 including a conductive layer 102 or pattern formed in the base layer 101 or another a substrate. The conductive layer 102 may be formed within or on the base layer 101 using any combination of methods, for example, by laser ablating the electrically insulating material (an insulating layer 103) of the base layer 101 to expose the electrically conductive material underneath, inserting conductors with physical attachment to a control circuit, electroplating and/or screen-printing a conductive material on top of an insulating material, or any other methods can be used to dispose the conductive layer 102 on the base layer 101. The base layer 101 can be composed of an electrically insulating material having a thickness sufficient to provide structural support to the components of the biosensor 100 (e.g., the conductive layer 102).

In some embodiments, the conductive layer 102 can be formed from a combination of thin-film metal, non-noble metal, and/or non-noble metal alloy to form one or more electrodes 104, with a thickness ranging from nanometers to micrometers. The use of thin film metal, non-noble metal, and/or alloy electrodes 104 can be designed to provide characteristics for reagent-free measurement of hematocrit and/or hemoglobin. For example, the reagent-free biosensor 100 can include electrodes 104 constructed from thin-film metal or non-noble metal alloy, such as Nickel, Silver, Stainless Steel, Palladium, Gold, Platinum, Carbon, Aluminum, Nickel-Chrome, Copper, Indium Zinc Oxide, Indium Tin Oxide, Tungsten, Ruthenium, and Graphene. In some embodiments, the electrodes 104 are not covered with inert coating, and can be used to measure hematocrit values of samples. The electrodes 104 can be designed with a single conductive material or using different conductive materials for different electrodes 104. The type of thin-film electrode substrate material for the electrodes 104 can be important to ensure accuracy over a wide range of hematocrit or hemoglobin levels and product stability. For example, sheet resistivity of the electrode 104 material can be an important characteristic of the thin film, enabling measurements at very low voltage across the electrodes 104 to further improve accuracy and precision.

In some embodiments, the conductive layer 102 can include a plurality of electrodes 104 disposed within/on base layer 101 near a proximal end (the end of the biosensor 100 in which a blood sample is applied to the test strip) of the biosensor 100. For example, the biosensor 100 can include two, three, four, or more electrodes 104 at or near the proximal end. The electrodes 104 can include a combination of electrode types, including but not limited to an anode, cathode, etc. Similarly, different electrodes 104 can be designed with different sizes, shapes, thickness, etc. to yield desired functionality. For example, the electrodes 104 can be constructed from thin-film metal, non-noble metal, and/or non-noble metal alloy substrate. In some embodiments, the plurality of electrodes 104 can be uniform in shape, size, and/or thickness.

In some embodiments, the conductive layer 102 can include a plurality of electrical strip contacts 106 disposed within/on the base layer 101 positioned at or near a distal end (the end of the biosensor 100 in which a blood sample is applied to the test strip) of the biosensor 100. For example, the biosensor 100 can include two, three, four, or more electrical contacts 106 at or near the proximal end. The strip contacts 106 can be used to exchange electricity, information, etc. with a test meter, as discussed in greater detail herein. Similar to the electrodes, the electrical strip contacts 106 can be constructed from thin-film metal, non-noble metal, and/or non-noble metal alloy substrate. In some embodiments, there can be different sets of contacts 106 for different functions. For example, the biosensor 100 can include a first and a second plurality of electrical contacts 106 corresponding to electrical contacts in the meter. Continuing the example, a current flow through the first plurality of electrical contacts 106 can cause the meter to wake up and enter an active mode while the meter can read code information provided through the second plurality of electrical contacts 106. The code information can then be used to identify, for example, the particular test to be performed, or a confirmation of proper operating status. In addition, the meter can also identify the inserted strip as either a test strip or a check strip based on the particular code information. In some embodiments, the biosensor 100 can include a plurality of conductive traces 108 electrically connecting the electrodes 104 to the plurality of electrical strip contacts 106.

In some embodiments, the biosensor 100 can also be designed with use of inert coatings or other materials, as shown in FIG. 1B and FIG. 1C. For example, the biosensor 100 can include an inert coating 111 on at least a portion of the conductive layer 102, the electrodes 104 (e.g., within the electrodes of the capillary chamber), the contacts 106, etc. to provide stabilization. The inert coating 111 can be applied across all the conductive layer 102, over a particular subset of the conductive layer 102 (e.g., all or part of the electrodes 104), and/or different inert coatings can be applied to different electrodes 104 to yield desirable results. For example, an inert coating can stabilize the surface by preventing redox species from contaminating the surface. The inert coating 111 may contain any combination of inert materials. For example, the inert coating can include organic and/or inorganic polymers, surfactants, anti-foaming agents, and/or wetting agents.

In some embodiments, the electrodes 104 can be modified to further stabilize the biosensor 100. For example, surface modifications to the electrodes 104 may include, but not limited to, plasma, corona treatment and/or UV treatment. A combination of the inert coating(s) and/or surface modification(s) may partially or fully cover the electrodes 104. In addition, inertly coated or surface modified electrodes 104 may offer a wider selection of electrode choices in hematocrit (HCT) due to improved performance characteristics, such as improved strip stability or shelf life. The surface modifications to the electrodes 104 can be provided across all the electrodes 104, over a particular subset of the electrodes 104, and/or different surface modifications can be applied to different electrodes 104 to yield desirable results.

In some embodiments, the biosensor 100 can include one or more spaces or distances between the plurality of electrodes 104 to measure the resistivity of blood therebetween. For example, the one or more spaces can be between the proximal and the distal electrodes for measuring hematocrit levels and may include distances for optimal performance, for example, between about 1 mm and about 3 mm. In some embodiments, the biosensor 100 can include a spacer 112 situated over the conductive layer 102. The spacer 112 can be a thin layer, constructed from an inert material, and/or have an inert coating. The inert spacer 112 may contain any combination of inert materials/coatings. For example, the inert spacer 112 can include organic and/or inorganic polymers, surfactants, anti-foaming agents, and/or wetting agents. The spacer is a separate layer from the insulating layer and can create the channel for the blood sample only.

In some embodiments, the biosensor 100 can include capillary channel 110 or chamber designed to receive a blood sample. The capillary channel 110 can include an open area that exposes at least a portion of the electrodes 104 and space/spacers such that a current can be applied (via the electrodes 104) through a sample (e.g., blood) received within the capillary channel 110. The applied electricity/current can be used to measure a level of resistivity/conductivity of the sample to be used in calculating a hematocrit level, as discussed in greater detail herein. In some embodiments, the biosensor 100 can include a coating or cover 113 as part of the capillary channel 110 for receiving blood samples to be measured. The combination of the thin-film base layer 101 with the inert spacer 112 and cover material can define the overall dimension of the capillary channel 110 port for blood entry. The capillary channel 110 may be dimensioned so as to be able to draw the blood sample in through the first opening, and to hold the blood sample in the capillary channel 110, by capillary action. In some embodiments, the biosensor 100 can include a tapered section that is narrowest at the proximal end or can include other indicia in order to make it easier for the user to locate the first opening and apply the blood sample. The capillary channel 110 and biosensor 100 can be formed using materials and methods described in U.S. Pat. No. 6,743,635, which is herein incorporated by reference in its entirety.

In some embodiments, the biosensor 100 can include an embedded code relating to data associated with a lot containing a plurality of the biosensor 100 test strips, or data particular to that individual biosensor 100. Such coded biosensor 100 (test strips) are further described in U.S. Pat. Pub. No. 3007/0015286, which is herein incorporated by reference in its entirety. In some embodiments, a calibration code can be included on the biosensor 100. The calibration code can be included on the biosensor 100 in the form of a second plurality of electrical strip contacts 106 near the distal end of the biosensor 100. The second plurality of electrical contacts 106 can be arranged such that they provide, when the biosensor 100 is inserted into the meter, a distinctly discernable calibration code specific to the lot that the biosensor 100 is from and is readable by the meter. The readable code can be read as a signal to access data, such as calibration coefficients, from an on-board memory unit in the meter related to biosensors 100 from that lot, or even information corresponding to individual biosensors 100.

The different components of the biosensor 100 can be formed using any combination of methods known in the art. For example, the biosensor 100 can be created by forming multiple layers using a fill dielectric, etching, sputtered, electroplating, etc.

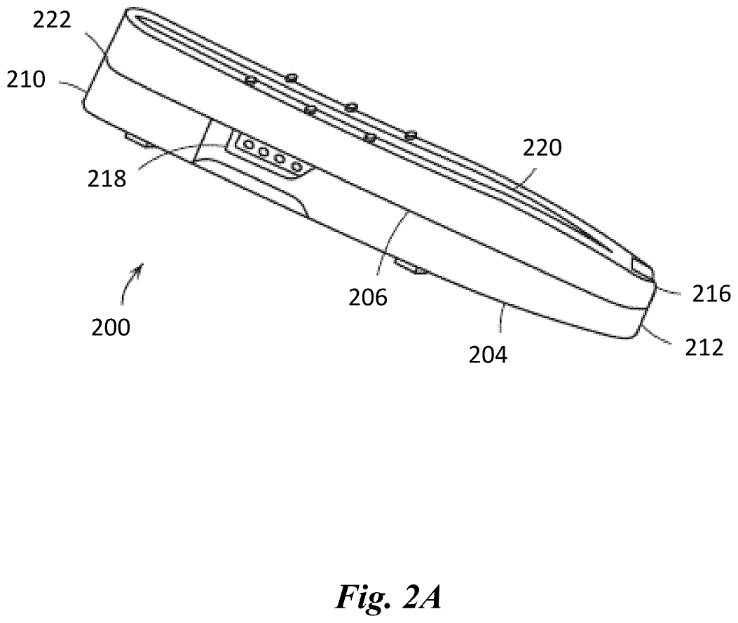

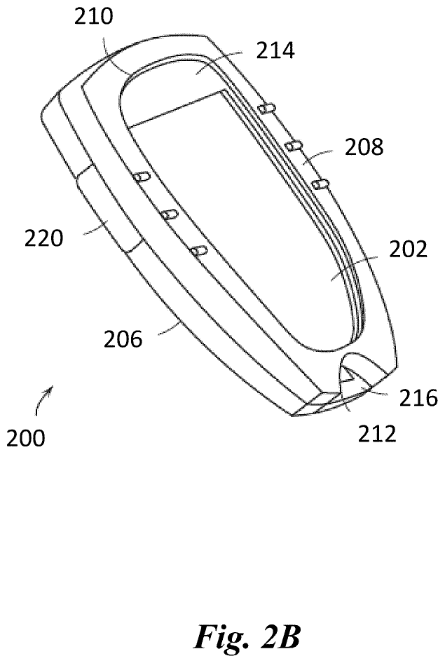

FIG. 2A and FIG. 2B illustrate an example illustration of a meter 200 that can be used to measure a hematocrit and estimate a hemoglobin level in a blood sample on the biosensor 100. The meter 200 can include a housing having a test port for receiving a distal end of the biosensor 100 (or test strip), making an electrical connection with the contacts 106, and a processor or microprocessor programmed to perform methods and algorithms to determine hematocrit/hemoglobin concentration in a test sample or control solution as disclosed in the present disclosure. In some embodiments, the meter 200 can have a size and shape to allow it to be conveniently held in a user's hand while the user is performing the hematocrit and estimate a hemoglobin measurement. The meter 200 may include a front side 202, a back side 204, a left side 206, a right side 208, a top side 210, and a bottom side 212. The front side 202 may include a display 214, such as a liquid crystal display (LCD). A bottom side 212 may include a strip connector 216 into which biosensor 100 can be inserted to conduct a measurement. The meter 200 may also include a storage device for storing test algorithms or test data. The left side 206 of the meter 200 may include a data connector 218 into which a removable data storage device 220 may be inserted, as necessary. The top side 210 may include one or more user controls 222, such as buttons, with which the user may control meter 200, and the right side 208 may include a serial data connector (not shown). In some embodiments, the meter 200 can include a decoder for decoding a predetermined electrical property, e.g. resistance, from the biosensor 100s as information. The decoder operates with, or is a part of, the microprocessor.

In some embodiments, the meter 200 can be used in combination with the biosensor 100 to measure a hematocrit (HCT) level in a blood sample. For example, an electrical current can be applied across the thin reagent-free electrodes 104 to obtain an electrical measurement, such as an AC impedance at a plurality of frequencies, through a sample. In some embodiments, all the electrodes on the test strip are reagent-free, such that all measurement performed without a reagent. The HCT measurement sequence can begin after a drop of blood or a control signal is detected when the drop completes the circuit between the HCT measurement a proximal and distal electrodes 104. In some embodiments, the hematocrit measurement sequence can be initiated only when the meter 200 detects a full sample capillary chamber 110. After the drop is detected or the capillary chamber 110 is full, an excitation voltage signal can be applied through the HCT electrodes 104, for example a proximal and distal electrode. The electrodes 104 can be designed such that only a low voltage is required to measure a hematocrit level, for example, less than 100 mv. The salt content of blood creates an electronic signature, in which the magnitude and phase response can be mapped to the HCT of the blood.

Various systems and methods can be used for measuring the HCT concentration from step response to impedance measurement. In some embodiments, a method of measuring the HCT for a meter 200 can include using multiple setpoints of relatively high frequency (10 kHz-500 kHz) magnitude and phase measurements to measure the HCT of the applied blood sample. In some embodiments, the phase measurement is done using narrow time pulse measurements that can be accumulated over a sample window. The impedance of the electrical signature can be affected by temperature, so the true HCT reading can be corrected for temperature for the temperature difference from 24° C. (dT). A method of measuring the HCT for the meter 200 can mix analog and digital circuitry to measure the HCT complex impedance (HCT impedance magnitude and phase). The meter 200 can use any combination of circuitry and measurement methods for measuring a HCT level in blood, such as for example, as discussed in U.S. patent application Ser. No. 16/787,417, incorporated here by reference in its entirety.

The biosensor 100 of the present disclosure can be used to measure hematocrits values with reagent free or inert coated electrodes 104. In some embodiments, the meter 200 can determine a hemoglobin concentration from the HCT measurement. The hemoglobin concentration can be converted directly from percent HCT using any combination of methods known in the art. For example, the measured HCT level can be divided by a factor of three to determine a hemoglobin level in the sample. For example, a look-up table can be used based on the measured HCT level to find the corresponding hemoglobin level. This look-up table can be stored in the meter, or the meter can communicate with an external computer or other processing device that include the look-up table stored thereon. Using the meter 200 and the biosensor 100 in combination can be used to measure an HCT level and hemoglobin level in a sample without the use of reagents.

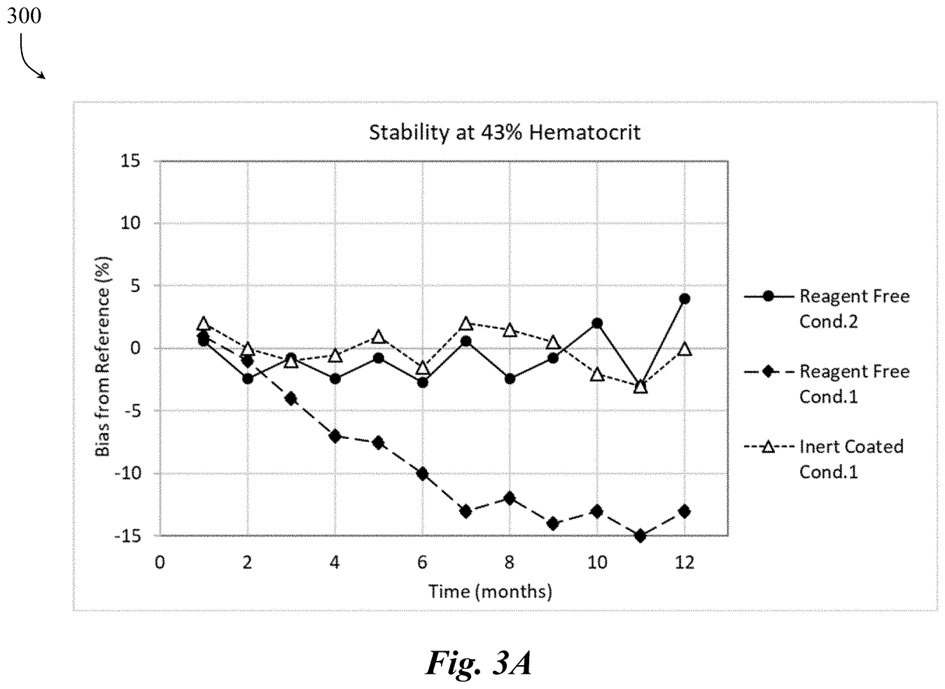

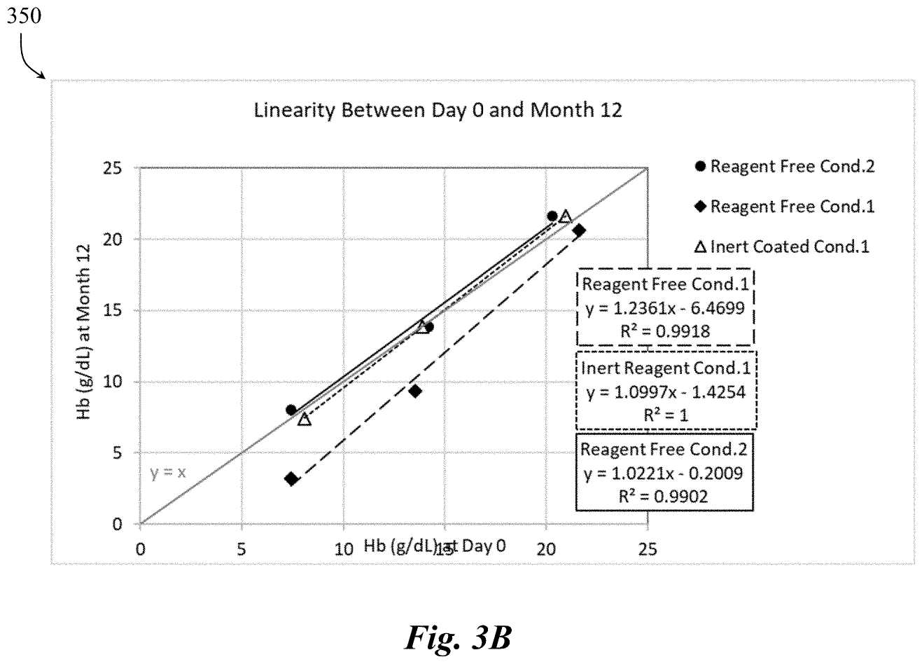

Referring to FIGS. 3A and 3B, example benefits of using the biosensor 100 design discussed with respect to FIG. 1A are depicted. As shown in FIGS. 3A and 3B, in certain combinations of electrode 104 substrate type and/or strip storage conditions, the biosensor 100 (or test strip) performance characteristics, such as strip stability, can significantly improve with inert coating and/or electrode surface modification. This improvement can increase the amount of compatible electrode 104 substrates for the biosensor 100. FIG. 3A depicts a chart 300 showing hematocrit bias from reference device over one-year period. The y-axis in chart 300 represents a percentage of bias from the reference and the x-axis represent the progression of time from 0 months to 12 months. To obtain the data from chart 300, reagent-free and inertly coated test strips that were stored with desiccant (Cond.1) or without desiccant (Cond.2), as reflected by the lines in the chart. As shown in chart 300, the stability performance of reagent free strips may be adversely affected when stored under Condition 1, as represented by the diamonds in the graph showing that after 12 months of storage, the hematocrit recovery was reduced by 14% HCT points. However, the stability performance of strips stored under the same condition can be improved by adding an inert coating to the test strip, as represented by the triangles in the graph showing an average bias of 0.1% HCT points throughout stability. Under other storage conditions, the reagent free test strips can be very stable, as represented by the circles in the graph which shows an average bias of 0.7% HCT points throughout stability.

FIG. 3B depicts a chart 350 showing a linearity response between hemoglobin results from day 0 and month 12. The y-axis in chart 350 represents hemoglobin results at month 12 and the x-axis represent hemoglobin results at month 0. Similar to chart 300, the data in chart 350 is based on reagent-free and inertly coated test strips that were stored with desiccant (Cond.1) or without desiccant (Cond.2), as reflected by the lines in the chart. As shown in chart 350, the reagent free test strips without desiccant performed similarly to the inert coated test strips with desiccant, whereas the reagent free test strips with desiccant, had a different result, demonstrating that an inert coating can provide improved stability over time across a wide range of hemoglobin levels from 7 g/L-20 g/dL.

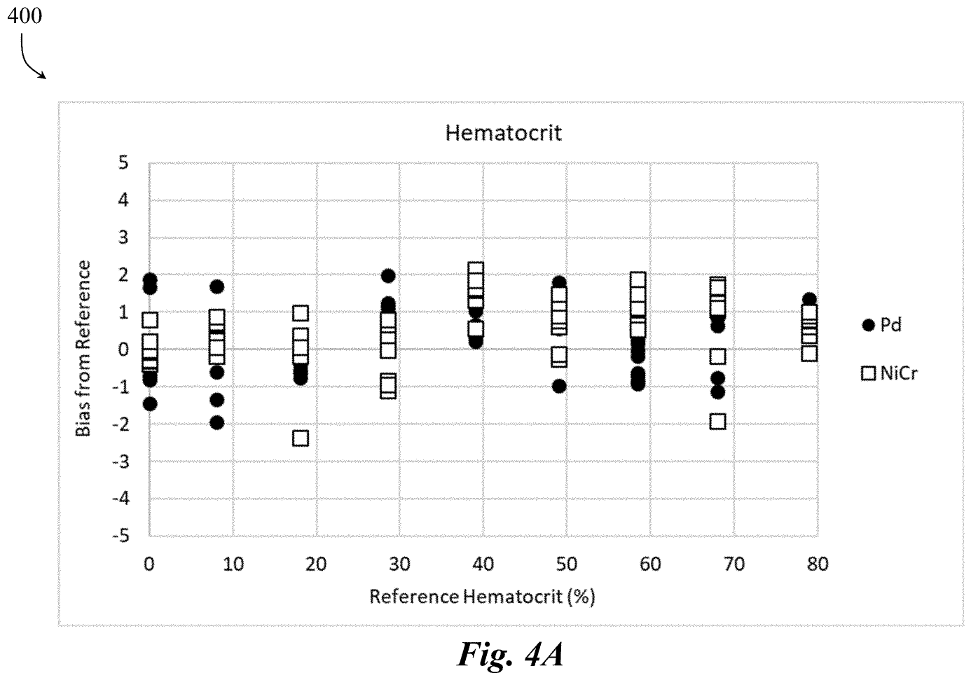

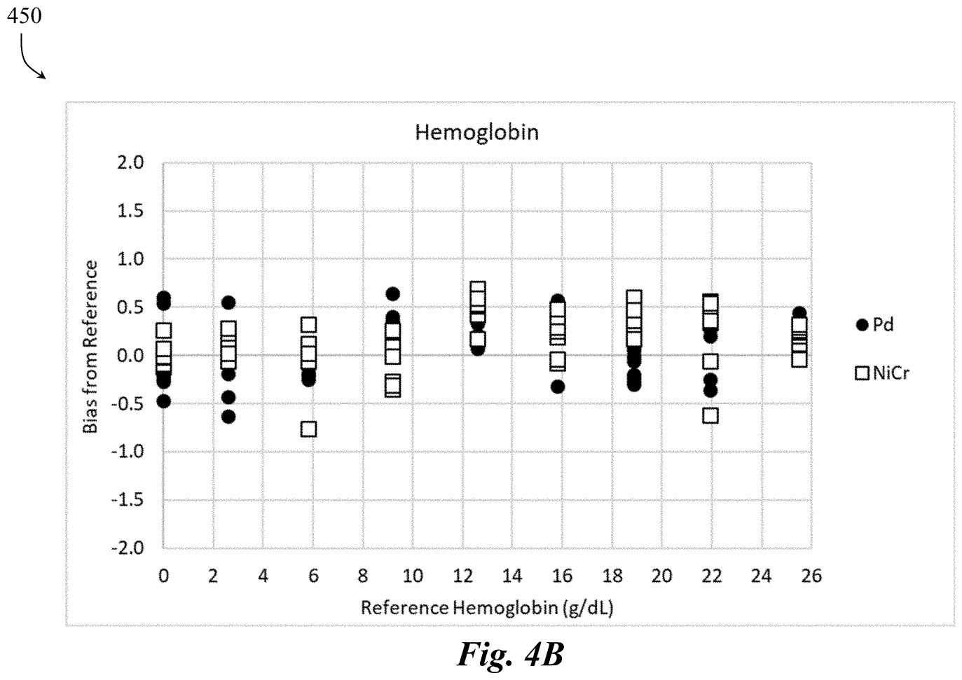

Referring to FIGS. 4A and 4B, in some embodiments, the relationship between the AC or DC response and hematocrit and/or hemoglobin can be determined through mathematical functions and then plotted against a reference device. The charts 400, 450 provide examples of the thin-film electrodes 104 may include palladium and alloy containing nickel-chrome, utilizing DC or AC voltage measurements. Chart 400 shows the plotted AC or DC response for hematocrit determination with Palladium (Pd) and alloy containing Nichrome (NiCr) and chart 450 shows the plotted AC or DC response for hemoglobin determination with Palladium (Pd) and alloy containing Nichrome (NiCr). The y-axis in chart 400 represents a percentage of bias from the reference and the x-axis represent the reference hematocrit. The y-axis in chart 450 represents a percentage of bias from the reference and the x-axis represent the reference hemoglobin. The results of the chart 450 demonstrate accurate and precise HCT and Hb recovery, within ±2.5% HCT and ±0.7 g/dL, respectively.

In operation, in some embodiments, the biosensor 100 can be used with the meter 200 for measuring hematocrit and/or hemoglobin within a blood sample. The meter 200 for measuring hematocrit and/or hemoglobin can include a portable, handheld device, for example, the meter 200 as discussed with respect to FIGS. 2A and 2B, and can be designed to measure hematocrit and/or hemoglobin levels without using a reagent. The biosensor 100 design of the present disclosure can work without the use of reagents while other designs require the use of reagents because the biosensor 100 is designed to specifically measure Hematocrit, whereas other test strips measure Hemoglobin, which require the use of a reagent. For example, the biosensor 100 of the present disclosure can obtain a Hematocrit measurement via conductivity, which does not require the use of a reagent. To have an effective electrical measurement for Hematocrit, be it conductivity or impedance, when using a biosensor 100, disposable test strip, etc. the biosensor 100 must have very consistent electrical properties. The use of the materials, strip design and production methods to produce thin film electrodes synergistically support uniform electrical performance. Additionally, the sheet resistivity can be maintained to high uniformity which means the key electrical parameters such as contact resistivity to the meter 200, capacitance and electrode impedance are uniform between biosensor 100 (or test strips).

Typically, in operation, a user purchases biosensor (e.g., test strips) that interface with the meter 200. For example, the user can purchase a biosensor 100 discussed with respect to FIGS. 1A-1C. The biosensor 100 can include thin film electrodes 104 formed from at least one of noble metal, non-noble metal alloys, non-metal that are reagent free and/or inert coated. The user can draw a tiny amount of blood (a few microliters or less) from a finger or other area, for example, using a lancet and a blood droplet is applied onto the exposed end of the biosensor 100 (e.g., proximal to the capillary chamber 110) which has an open port for the blood. The user can also draw blood from another human or non-human subject. Thereafter, the biosensor 100 with the sample thereon can be inserted into a test meter 200, for example, proximal end first. In some embodiments, the meter 200 may apply a fill-detect voltage between fill-detect electrodes on the meter 200 and/or biosensor 100 to measure any resulting current flowing between the fill-detect electrodes. If this resulting current reaches a sufficient level within a predetermined period of time, the meter 200 can indicate to the user that adequate sample is present (e.g., on a display or other indicator).

When an adequate sample is received, the biosensor 100 can be inserted into the meter 200 connector port and a resistivity/conductively of the sample can be measured by applying an electrical current through the sample (e.g., via the electrodes 104) to determine the hematocrit level in units of g/dL or mmol/L, depending on regional preferences.

In a typical system, a level of resistance/capacitance of the blood can be measured by applying a current to a working electrode (e.g., the proximal electrode) that is in contact with the sample to be analyzed. An electrical circuit can be completed through a counter electrode (e.g., the distal electrode) that is also in contact with the sample. In accordance with the present disclosure, the determination of hematocrit on the reagent-free, thin-film biosensor 100 does not require use of a reagent and can be used with a variety of common techniques that are driven by very low voltages to provide very accuracy and precise measurements. For example, techniques such as AC impedance, a DC charging current, conductivity, etc. Some techniques are more advantageous than others, with respect to minimizing potential interference effects of electrolytes (i.e. sodium), proteins, lipids, and temperature.

The use of these thin film electrode sensors enable accurate, precise, and consistent (between each sensor test strip) low voltage, fast, and stabilized electrical measurements, which provides significant advantages over either optical (with or without reagents) and standard electrical measurements. For example, optical measurements, with or without active reagent, are subject to optical interference from other components in the blood that will absorb or scatter the optical signal. Endogenous materials such as bilirubin and lipid micelles are common sources of optical interference. In addition, exogenous substances, such as pharmaceuticals can impact the optical characteristics of the blood sample. For electrical measurements, the surface area of the electrode is a critical parameter in the determination of Hct, such that systems incorporating reusable electrodes can be subject to protein deposition on the surface of the electrode. Even with protease cleaning, it is common for residual material to remain deposited of the electrode surfaces thus altering the available surface area over time.

Lastly, single use electrodes system, not using the uniform thin film electrodes of the biosensors 100 described in this disclosure, by their very size and production methods are susceptible to surface area variations and a requirement for high assay voltages to achieve suitable measurement performance. At higher voltages electrochemical (Redox) reactions can occur with both endogenous and exogenous materials in the blood, (vitamin C and aspirin respectively are examples of material easily oxidized). Therefore, the use of a single use thin film electrode designed biosensor 100 provides consistent electrical conductively that is not subject to degradation in existing system. The performance characteristics of the biosensor 100, such as accuracy, precision, and/or stability, may be dependent on the type of thin film electrode substrate, strip storage conditions, and/or presence of an inert coating.

In short, using the biosensor 100 of the present disclosure to measure the resistance/capacitance of the blood sample, a hematocrit measurement can be determined. Thereafter, a hemoglobin measurement can be derived, by the meter 200, using the hematocrit measurement, for example, by dividing the hematocrit level by a factor of three. The results can then be provided to the user via a display on the meter 200. As a result, the combination of the biosensor 100 and the meter 200 can use a thin film electrodes to determine a hematocrit and hemoglobin measurement from a blood sample.

In some aspects, the present disclosure provides a device for the measurement of hematocrit in a blood sample, the device comprising: a conductive pattern formed within a substrate, the conductive pattern being formed from a thin film material; a spacer deposed on the conductive pattern; and a capillary chamber exposing at least a portion of the conductive pattern and for receiving the blood sample. The conductive pattern can include a plurality of contacts for communicating with a test meter and a plurality of electrodes for electrically measuring the blood sample. The blood sample can be measured by applying an AC impedance at a plurality of frequencies. In some embodiments, the blood sample is measured by applying a low voltage less than 100 mv signal across a plurality of electrodes. The low voltage can be designed to determine the hemoglobin concentration in the blood sample that makes use of a known correlation of the hemoglobin concentration to a hemoglobin relationship to determine a hemoglobin value.

In some embodiments, the plurality of electrodes are uniform thin film electrodes. In some embodiments, the thin film electrodes have a thickness of nanometer to micrometer. The plurality of electrodes can be reagent free or have an inert coating. In some embodiments, the inert coating partially coats or fully coats the plurality of electrodes. The inert coating can include at least one of a surfactant and/or a polymer. In some embodiments, the conductive pattern is composed of a combination of a noble metal, a non-noble metal alloys, and a non-metal. In some embodiments, the device further comprises a distance between proximal and distal electrode(s) from 0.5-5.5 mm. In some embodiments, the blood sample is one of a human blood sample and a non-human blood sample.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the disclosure. It is intended that the present disclosure be limited only to the extent required by the appended claims and the applicable rules of law.

It is also to be understood that the following claims are to cover all generic and specific features of the disclosure described herein, and all statements of the scope of the disclosure which, as a matter of language, might be said to fall therebetween.

What is claimed is:

1. A test strip comprising:
a conductive pattern formed on a substrate, the conductive pattern being formed from a thin film material, the conductive pattern comprising:
a plurality of electrodes configured to perform a reagent-free measurement of hematocrit levels in a blood sample;
a plurality of conductive contacts configured to communicate with a test meter; and
a plurality of conductive traces configured to electrically connect the plurality of electrodes to the plurality of conductive contacts;
an inert layer positioned on at least a portion of the conductive pattern; and
a capillary chamber exposing at least a portion of the plurality of electrodes, the capillary chamber being defined by the inert layer for receiving a blood sample and delivering the blood sample to the plurality of electrodes.

2. The test strip of claim 1, wherein the plurality of electrodes are uniform thin film electrodes.

3. The test strip of claim 1, wherein the plurality of electrodes has a thickness in a range of 10 nm (100 Å) to 3,000 nm (3 μm).

4. The test strip of claim 1, wherein the plurality of electrodes has a thickness in a range of 20 nm (200 Å) to 1,000 nm (1 μm).

5. The test strip of claim 1, wherein the plurality of electrodes has a thickness in a range of 30 nm (300 Å) to 60 nm (600 Å).

6. The test strip of claim 1, wherein the plurality of electrodes are formed from a thin film of a non-noble metal.

7. The test strip of claim 1, wherein the plurality of electrodes includes a proximal electrode and a distal electrode, wherein a distance between proximal electrode and the distal electrode is in a range from 0.5 mm-5.5 mm.

8. The test strip of claim 1, wherein the inert layer fully coats the plurality of electrodes.

9. A system for measuring hematocrit in a blood sample, the system comprising:
a test strip comprising: a conductive pattern formed on a substrate, the conductive pattern being formed from a thin film material, the conductive pattern comprising a plurality of electrodes configured to perform a reagent-free measurement of hematocrit levels in a blood sample; a plurality of conductive contacts; and a plurality of conductive traces configured to electrically connect the plurality of electrodes to the plurality of conductive contacts; an inert layer positioned on at least a portion of the conductive pattern; a capillary chamber exposing at least a portion of the plurality of electrodes, the capillary chamber being defined by the inert layer for receiving a blood sample and delivering the blood sample to the plurality of electrodes; and
a test meter configured to accept the test strip and to connect to the plurality of conductive contacts to determine a level of hematocrit in the blood sample received on the test strip.

10. The system of claim 9, wherein the test meter is configured to apply an AC impedance at a plurality of frequencies across the plurality of electrodes.

11. The system of claim 9, wherein the test meter is configured to apply a low voltage less than 100 mv signal across the plurality of electrodes.

12. The system of claim 11, wherein the test meter is further configured to determine a hemoglobin value from the level of hematocrit in the blood sample.

13. The system of claim 9, wherein the plurality of electrodes are uniform thin film electrodes.

14. The system of claim 9, wherein the plurality of electrodes has a thickness in a range of 10 nm (100 Å) to 3,000 nm (3 μm).

15. The system of claim 9, wherein the plurality of electrodes has a thickness in a range of 20 nm (200 Å) to 1,000 nm (1 μm).

16. The system of claim 9, wherein the plurality of electrodes has a thickness in a range of 30 nm (300 Å) to 60 nm (600 Å).

17. The system of claim 9, wherein the plurality of electrodes are formed from a thin film of a non-noble metal.

18. The system of claim 9, wherein the plurality of electrodes includes a proximal electrode and a distal electrode, wherein a distance between proximal electrode and the distal electrode is in a range from 0.5 mm-5.5 mm.

19. The system of claim 9, wherein the inert layer fully coats the plurality of electrodes.

20. A method for determining a hematocrit value in a blood sample, the method comprising:
applying, by a test meter, an electrical current across a plurality of electrodes on a test strip, wherein the test strip comprises a conductive pattern formed on a substrate, the conductive pattern being formed from a thin film material, the conductive pattern comprising the plurality of electrodes configured to perform a reagent-free measurement of hematocrit levels in a blood sample; a plurality of conductive contacts; and a plurality of conductive traces configured to electrically connect the plurality of electrodes to the plurality of conductive contacts; an inert layer positioned on at least a portion of the conductive pattern; a capillary chamber exposing at least a portion of the plurality of electrodes, the capillary chamber being defined by the inert layer for receiving a blood sample and delivering the blood sample to the plurality of electrodes;
measuring, by the test meter, conductivity of the blood sample; and
calculating, by the test meter, a hematocrit value of the blood sample based on the conductivity of the blood sample.

21. The method of claim 20 further comprising determining, by the test meter, a hemoglobin value of the blood sample from the hematocrit value.
